(19) United States
(12) Patent Application Publication
(10) Pub. No.: US 2007/0168021 A1
(43) Pub. Date: Jul. 19, 2007

(54) Porous three dimensional nest scaffolding

(76) Inventors: David R. Holmes JR., Rochester, MN (US); Gurpreet S. Sandhu, Rochester, MN (US); Robert D. Simari, Rochester, MN (US)

Correspondence Address: QUARLES & BRADY LLP, 411 E. WISCONSIN AVENUE, SUITE 2040, MILWAUKEE, WI, 53202-4497, US

(21) Appl. No.: 11/333,054

(22) Filed: Jan. 17, 2006

(51) Int. Cl. A61F 2/06; A61F 2/82; A61F 2/02

(52) U.S. Cl. 623/1.42; 623/1.41; 623/23.76; 424/423; 435/399

(57) ABSTRACT

The invention provides porous three dimensional scaffold structures that may be used to deliver a bioactive agent, such as cells, into a location within the body. In one example form, the porous three dimensional structure may be a stent. In another example form, the porous three dimensional structure may be a microscale or nanoscale device for the delivery of the bioactive agent. The scaffold may include a substrate and one or more nests connected to the substrate. The nest(s) extend away from the substrate to define an enclosed volume on the substrate within each nest. The nests have openings that extend from an outer surface of the nest to the enclosed volume within each nest. The scaffold includes a bioactive agent disposed within the enclosed volume of at least one nest on the substrate. The bioactive agent is delivered to the patient when the scaffold is located within the patient.

### CROSS-REFERENCES TO RELATED APPLICATIONS

Not Applicable.

### STATEMENT REGARDING FEDERALLY SPONSORED RESEARCH

Not Applicable.

### BACKGROUND OF THE INVENTION

1. Field of the Invention

The present invention relates to porous three dimensional structures that may be used to deliver one or more bioactive agents into a location within the body. In one example form, the porous three dimensional structure may be a stent. In another example form, the porous three dimensional structure may be a microscale or nanoscale device for the delivery of the bioactive agent.

2. Description of the Related Art

The narrowing and the occlusion of the coronary arteries are major causes of heart disease. Coronary artery disease can lead to ischemia perfusion defects and/or myocardial infarction. Coronary artery disease is quite common and therefore, many diagnostic and treatment methods have been proposed to diagnose and/or treat coronary artery disease.

Regarding examples of coronary artery disease treatment methods, stents are commonly used in situations where part of a blood vessel wall or stenotic plaque blocks or occludes blood flow in the vessel. Stents are typically implanted within a blood vessel in a contracted state, and expanded once in place in the blood vessel to allow fluid flow through the vessel and the stent. Such a stent can be moved along a guide wire previously placed in the vessel, and expanded by inflation of a balloon within the stent. Deflation of the balloon and removal of the guide wire leaves the stent in place in the vessel, locked in an expanded state. Example stents can be found in U.S. Pat. Nos. 6,752,826, 6,440,166, 6,224,626, 6,156,064, 6,120,535, 5,645,559, and 5,629,077. Other three dimensional prosthetic structures can be found in U.S. Pat. Nos. 6,520,997, 6,008,430, and 5,807,406. These patents and all other documents cited herein are incorporated herein by reference.

With respect to coronary artery disease diagnostic methods, nuclear imaging has been used for the detection of myocardial perfusion abnormalities. In another technique described in U.S. Pat. No. 5,961,459, hollow microcapsules are first administered into a blood vessel of a patient having a perfusion defect. If desired, an ultrasonic image is formed of the tissue, and the occlusion is at least partially removed such that the blood flow in at least one area of the tissue is increased, and an ultrasonic image of the tissue is obtained after treatment. This is based on the observation of the particular properties of the microcapsules in the myocardium. It is also the basis of providing appropriate drugs to that site.

However, there is still a need for structures that can be used to treat coronary artery disease or any other disease within the body.

### SUMMARY OF THE INVENTION

The invention provides porous three dimensional biomedical structures. In one example form, the structure is a scaffold for location in a patient. In another example form, the porous three dimensional structures can be configured as a radially expandable lumenal prosthesis (e.g. stent) for placement within a body lumen such as a blood vessel. The radially expandable lumenal prosthesis includes one or more nests for delivery of a bioactive agent into the body lumen. In yet another example form, the porous three dimensional structures can be configured as a microscale or nanoscale device for delivery of a bioactive agent to a vessel of a patient such as a body lumen or vascular structure.

The scaffold may include one or more substrate layers. Each layer has one or more nests. In one example form, the scaffold is a three dimensional structure made up of at least 3 layers that can be woven or non-woven. Each layer of the scaffold has regular but not necessarily uniform porosity with the pore size being at least 60 microns. Multiple layers can be joined by interlocking joints, hinges, or rivets with the three dimensional structure being flexible or locked. The nests allow for in growth or protection of cells, precursors, growth factors, drugs, etc. Preferably, but not necessarily, the nests are rigid to protect cells or other bioactive agents inside the nests. Also, regular, but not necessarily uniform, porosity of the nests is preferred. The nests may be regularly spaced on the substrate. The scaffold may comprise non-woven or non-fibrous materials.

The scaffold structure can be used in grafts, pledges, artificial ureters, shunts, cartilage, dura, tympanic membrane, biliary duct, skin, biological pacemaker wires, leads, heart valves, nerve fibers, aneurysm coils or stents. In one example form, the structure is used for intravascular devices, such as stents, etc. The advantages of this approach include the ease of manufacture, ability to obtain regular pore sizes, the ability to vary pore sizes, the ability to use different materials for the different layers.

Scaffold structures according to the invention may be used as microscale or nanoscale devices for delivery of a bioactive agent to a vessel of a patient. The microscale or nanoscale devices provide a micro environment to grow cells. Cells are seeded within the three dimensional structure. The microscale or nanoscale devices may be various shapes such as disc, elliptical, spheroid (ball), honeycomb, buckyball-like, or a sphere with regular or irregular (i.e., differently sized) depressions. Preferably, the microscale or nanoscale devices are 10-20 microns in size. The scaffold structure embolizes and the cells inside may make drugs in vivo. The microscale or nanoscale devices may be delivered locally by injection with a catheter or administered intravenously.

The invention also provides a method that is an alternative approach to a conventionally sized stent. In the method, scaffold structures according to the invention are injected upstream of a vessel closure (e.g. an occlusion). The structures pool by the vessel closure and in the capillaries in the area around the closure. The structures have bioactive agents (e.g., cells) seeded therein that promote angiogenesis in order to bypass the closure. Thus, the invention provides a method for treating an occlusion of a blood vessel in a patient.

Accordingly, in one aspect, the invention provides a scaffold for location in a patient. The scaffold includes a substrate and one or more nests connected to the substrate. The nest(s) extend away from the substrate to define an enclosed volume on the substrate within each nest. The nests have openings that extend from an outer surface of the nest to the enclosed volume within each nest. The scaffold includes a bioactive agent disposed within the enclosed volume of at least one nest on the substrate. The substrate of the scaffold may be a flexible mesh such that the bioactive agent and fluids may pass through openings in the substrate and such that the substrate of the scaffold may be formed into a variety of shapes. In one version, the openings of the substrate are least 60 microns. In one embodiment, the scaffold has a plurality of nests. The nests may be rigid to protect cells inside the nests. The nests may be regularly spaced on the substrate. In one version, the openings of the nest are least 60 microns. In one form, the substrate is formed from polymeric mesh, and each nest is formed from polymeric mesh. In another form, the substrate is formed from metallic wire cloth, and each nest is formed from metallic wire cloth.

In one form, the nest(s) include a side wall and a top wall wherein the side wall and/or the top wall of the nest have openings such that the bioactive agent and fluids may pass through openings. When the scaffold is a two layer scaffold, the top wall of the nest may be formed by a part of a second substrate spaced apart from the first substrate. The nest connected to the second substrate extends away from the second substrate to define an enclosed volume on the second substrate associated with the nest connected to the second substrate. The nest connected to the second substrate has openings that extend from an outer surface of the nest to the associated enclosed volume, and a bioactive agent is disposed within the enclosed volume on the second substrate. The nest connected to the second substrate may include a side wall and a top wall. When the scaffold is a three layer scaffold, the top wall of the nest connected to the second substrate may be formed by a part of a third substrate spaced apart from the second substrate.

In another aspect, the invention provides a microscale or nanoscale device for delivery of a bioactive agent to a vessel of a patient. The device is a microscale or nanoscale scaffold according to the invention.

In one application of the microscale or nanoscale device according to the invention, an occlusion of a blood vessel in a patient is treated by injecting a plurality of the microscale or nanoscale devices upstream of the occlusion such that the plurality of the devices locate near the occlusion and release a bioactive agent.

In another application of the microscale or nanoscale device according to the invention, an occlusion of a blood vessel in a patient is treated by injecting a plurality of magnetic microscale or nanoscale devices in the blood vessel, and moving a magnetic field external to the patient such that the plurality of the devices are advanced to and locate near the occlusion where the devices release a bioactive agent.

In yet another application of the microscale or nanoscale device according to the invention, an occlusion of a blood vessel in a patient is treated by magnetically adhering a plurality of magnetic microscale or nanoscale devices to a guide wire, moving the guide wire in the blood vessel such that the plurality of the devices are located near the occlusion, and releasing the plurality of the devices from the guide wire wherein the devices release a bioactive agent near the occlusion.

In yet another application of the microscale or nanoscale device according to the invention, a bioactive agent is delivered to tissue in a patient by administering to a site in the patient a plurality of the microscale or nanoscale devices such that the plurality of the devices locate near the tissue. In one form, the devices include a targeting moiety that binds with a moiety of the tissue. The targeting moiety may be selected from ligands, antibodies, receptors, hormones, adhesion molecules, or portions or fragments thereof. Optionally, a magnetic field may be applied near the tissue such that the plurality of the devices locate near the tissue, or an electrical field may be applied near the tissue such that the plurality of the devices locate near the tissue.

In yet another aspect, the invention provides a radially expandable lumenal scaffold. The radially expandable lumenal scaffold includes a scaffold according to the invention. The scaffold is wrapped about an axis to form the radially expandable lumenal scaffold. Alternatively, the scaffold is folded toward an axis to form the radially expandable lumenal scaffold. In one form, each nest extends away from the substrate toward the axis. In another form, each nest extends away from the substrate and away from the axis.

These and other features, aspects, and advantages of the present invention will become better understood upon consideration of the following detailed description, drawings, and appended claims.

### BRIEF DESCRIPTION OF THE DRAWINGS

FIG. 1 is a top perspective view showing one embodiment of a scaffold according to the invention.

FIG. 2 is a side elevational view of an embodiment of a three layer scaffold according to the invention.

FIG. 3 is an end view of the scaffold of FIG. 1 rolled up for delivery to a body lumen.

FIG. 4 shows the stent of FIG. 3 in a body lumen having been expanded by inflation of a balloon located within the stent.

FIG. 5 is an end view of the scaffold of FIG. 1 folded inward for delivery to a body lumen.

FIG. 6 shows the stent of FIG. 5 in a body lumen having been expanded by inflation of a balloon located within the stent.

Like reference numerals will be used to refer to like parts from Figure to Figure in the following description of the drawings.

### DETAILED DESCRIPTION OF THE INVENTION

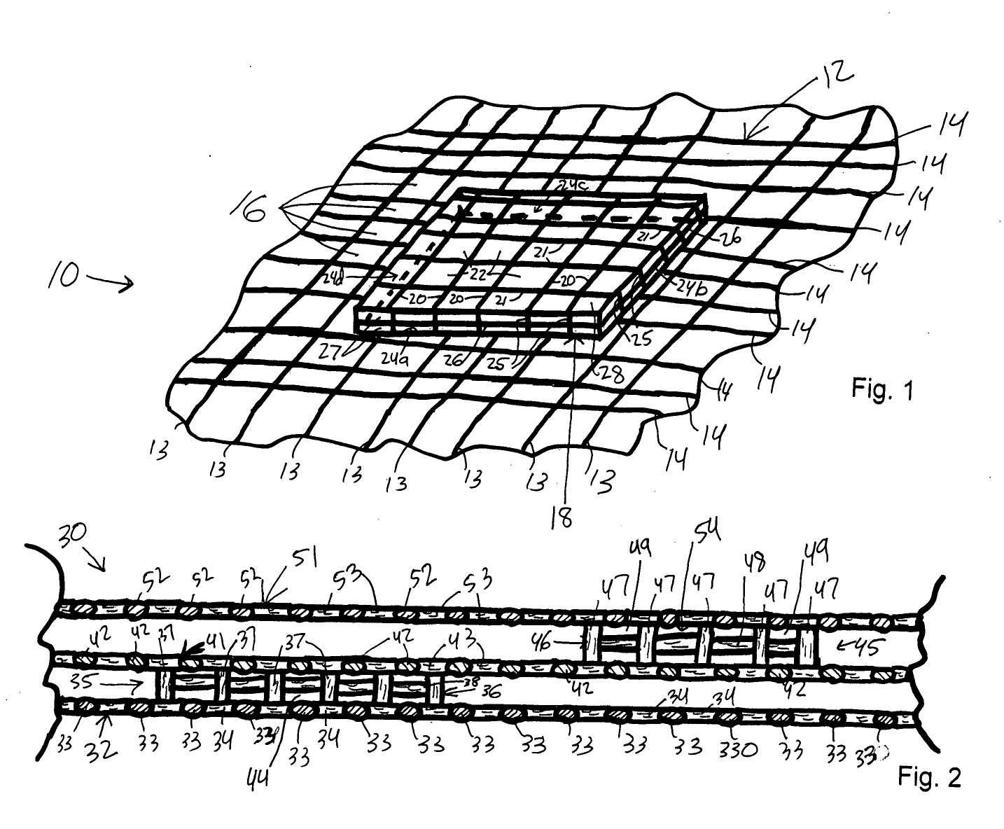

Turning to FIG. 1, there is shown a detailed view of one section of an example embodiment of a single layer scaffold according to the invention. The scaffold 10 includes a mesh substrate 12 formed from longitudinal strands 13 and lateral strands 14. The mesh substrate 12 has a plurality of openings 16 formed by the intersecting longitudinal strands 13 and lateral strands 14. The scaffold 10 also includes a nest 18 having a top wall 19 formed from longitudinal strands 20 and lateral strands 21. The top wall 19 of the nest 18 has a plurality of openings 22 formed by the intersecting longitudinal strands 20 and lateral strands 21. The nest 18 has side walls 24a, 24b, 24c, 24d formed from longitudinal strands 25 and lateral strands 26. The side walls 24a, 24b, 24c, 24d of the nest 18 have a plurality of openings 27 formed by the intersecting longitudinal strands 25 and lateral strands 26. The top wall 19 and the side walls 24a, 24b, 24c, 24d of the nest 18 define a rectangular enclosed volume 28 in the interior of the nest 18. The rectangular shaped nest 18 is merely one form of a nest, and other shapes (e.g., disc, sphere, oval, etc.) and sizes are also suitable depending on the application of the nest. FIG. 1 shows one section of the scaffold 10 with one nest 18. However, the scaffold 10 typically has a plurality of nests and preferably, the nests are regularly spaced on the mesh substrate 12.

The mesh substrate 12 of the scaffold 10 may be formed from a metallic, polymeric or composite material that is woven or non-woven. Preferably, the mesh substrate 12 is flexible so that the mesh substrate 12 may be formed into shapes suitable for location and/or implantation in a body lumen. In one example form, the mesh substrate 12 of the scaffold 10 is formed from a photosensitive polymeric material such as a polyimide using photolithographic techniques as described in U.S. Pat. No. 6,520,997. Such photolithographic techniques can be used to produce nanoscale devices. The mesh substrate 12 may also be formed from a bioresorbable material such as poly(lactide-glycolide), poly(propylene fumarate), poly(caprolactone), and poly(caprolactone fumarate). In another example form, the mesh substrate 12 of the scaffold 10 is formed from a metallic wire cloth such as a stainless steel wire cloth available from Belleville Wire Cloth Co., Inc., Cedar Grove, N.J., USA. This stainless steel wire cloth is purchased according to a mesh count, where mesh count is defined as the number of openings per linear inch laterally and longitudinally. Preferably, the mesh substrate of the metallic wire cloth has a mesh count of 80×80 or greater. A mesh count of 80×80 yields openings of a width of 0.006 inches (152 microns). Higher mesh counts (e.g., 325×325, 400×400) yield smaller openings.

The nest or nests 18 of the scaffold 10 may be formed from a metallic, polymeric or composite material that is woven or non-woven. Preferably, the nests 18 are rigid to protect materials inside the nest 18. In one example form, the nests 18 of the scaffold 10 are formed from a photosensitive polymeric material such as a polyimide using photolithographic techniques as described in U.S. Pat. No. 6,520,997. The nest or nests 18 of the scaffold 10 may also be formed from a bioresorbable material as described above. In another example form, the nests 18 of the scaffold 10 are formed from a metallic wire cloth as described above. The metallic wire cloth may be formed into the rectangular shape of the nest 18 of FIG. 1 using die pressing or any similar technique. Preferably, the openings 22, 27 of the nest 18 are least 60 microns. The nests 18 may be secured to the mesh substrate 12 using various techniques such as adhesive bonding, welding or heat sealing depending on the materials used for the nests 18 and the mesh substrate 12.

The enclosed volume 28 of one or more of the nests 18 may contain one or more bioactive agents for delivery within the body when the scaffold is implanted in a patient. A “bioactive agent” as used herein includes, without limitation, physiologically or pharmacologically active substances that act locally or systemically in the body. A bioactive agent is a substance used for the treatment, prevention, diagnosis, cure or mitigation of disease or illness, or a substance which affects the structure or function of the body or which becomes biologically active or more active after it has been placed in a predetermined physiological environment. Bioactive agents include, without limitation, cells, drugs, precursors, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, extracellular matrix and/or its individual components, pharmaceuticals, and therapeutics. The bioactive agent within each nest may be the same or different depending on the biological activity desired.

In one example application, the scaffold 10 may be located within a blood vessel to treat cardiovascular disease, and the bioactive agent contained within the nests 18 may be drugs delivered in microcapsule form that are seeded within the nests 18. Non-limiting examples of these types of cardiovascular drugs that can advantageously be delivered in microcapsule form include: (1) anti-platelet agents such as indobufen and ticlopidine hydrochloride; (2) thrombin inhibitors such as heparin; (3) fibrinolytic agents such as plasminogen activators, hementin, streptokinase and staphylokinase; (4) tissue factor inhibitors such as recombinant tissue pathway inhibitor; (5) vasodilators such as angiotensin-converting enzyme inhibitors; (6) calcium channel blockers such as verapamil and elgodipine; (7) potassium channel openers such as pinacidil and nicorandil; (8) anti-restenosis agents; (9) nitric oxide-scavengers or inhibitors of nitric oxide synthesis; (10) antioxidants or free radical-scavengers, e.g. for scavenging nitric oxide; and (11) antibiotics.

The mesh substrate 12 and/or the nests 18 of the scaffold 10 may include a coating covering all surfaces or specific surface regions of the mesh substrate 12 and nests 18. The coating may be adhered to, or deposited on, or adjacent the surface of the mesh substrate 12 and nests 18. The coating may be permanent or bioresorbable. The coating can be used to affect the physical properties of the mesh substrate 12 and nests 18. The coating can also be used for delivery of one or more bioactive agents. For instance, in one non-limiting example, a polymeric coating that releases heparin may be useful to inhibit platelet adhesion or reduce thrombogenicity. Of course, other suitable bioactive agents may be delivered from a polymeric coating. The coating thickness is selected depending on the activity desired.

The mesh substrate 12 and/or the nests 18 of the scaffold 10, or specific sections thereof, may be formed from one or more magnetic materials. The magnetic materials may be temporary magnetic materials or permanent magnetic materials. Some examples of suitable magnetic materials include: magnetic ferrite such as nanocrystalline cobalt ferrite; ceramic and flexible magnetic materials such as materials made from strontium ferrous oxide which is combined with a polymeric material; NdFeB; SmCo; and combinations of aluminum, nickel, cobalt, copper, iron, titanium as well as other materials. Also, materials such as stainless steel may be rendered sufficiently magnetic by subjecting the scaffold material to a sufficient electric and/or magnetic field such that the scaffold 10 or a section thereof is provided with magnetic properties. The mesh substrate 12 and/or the nests 18 may include one or more recesses which have the magnetic material contained therein. Alternatively, the mesh substrate 12 and/or the nests 18 may be coated on any or all surfaces with a coating which has magnetic properties. It is also possible to provide the mesh substrate 12 and/or the nests 18 with magnetic poles linearly arrayed in alternating polarity. The use of coating techniques could provide a first region with a first polarity, followed by a second region with a second polarity, followed by a third region of the first polarity, and so on, to create a linear array of alternating polarity regions.

Referring now to FIG. 2, there is shown a side elevational view of an embodiment of a multiple layer (i.e. three layer) scaffold according to the invention. The scaffold 30 includes a first mesh substrate 32 formed from longitudinal strands 33 and lateral strands 34. The first mesh substrate 32 has a plurality of openings formed by the intersecting longitudinal strands 33 and lateral strands 34. The scaffold 30 has a first nest 35 having side walls 36 formed from longitudinal strands 37 and lateral strands 38. The side walls 36 of the first nest 35 have a plurality of openings 39 formed by the intersecting longitudinal strands 37 and lateral strands 38. The scaffold 30 includes a second mesh substrate 41 formed from longitudinal strands 42 and lateral strands 43. The second mesh substrate 41 has a plurality of openings formed by the intersecting longitudinal strands 42 and lateral strands 43. The second mesh substrate 41 is spaced apart from the first mesh substrate 32 by the first nest 35. The first mesh substrate 32 and the second mesh substrate 41 and the side walls 36 of the nest 35 define a rectangular enclosed volume 44 in the interior of the first nest 35, the first mesh substrate 32 acting as the bottom wall of the first nest 35 and the second mesh substrate 41 acting as the top wall of the first nest 35. The rectangular shaped nest 35 is merely one form of a nest, and other shapes (e.g., disc, sphere, oval, etc.) and sizes are also suitable depending on the application of the nest.

The scaffold 30 has a second nest 45 having side walls 46 formed from longitudinal strands 47 and lateral strands 48. The side walls 46 of the second nest 45 have a plurality of openings 49 formed by the intersecting longitudinal strands 47 and lateral strands 48. The scaffold 30 includes a third mesh substrate 51 formed from longitudinal strands 52 and lateral strands 53. The third mesh substrate 51 has a plurality of openings formed by the intersecting longitudinal strands 52 and lateral strands 53. The third mesh substrate 51 is spaced apart from the second mesh substrate 41 by the second nest 45. The second mesh substrate 41 and the third mesh substrate 51 and the side walls 46 of the second nest 45 define a rectangular enclosed volume 54 in the interior of the second nest 45, the second mesh substrate 41 acting as the bottom wall of the second nest 35 and the third mesh substrate 51 acting as the top wall of the second nest 45. The rectangular shaped nest 45 is merely one form of a nest, and other shapes (e.g., disc, sphere, oval, etc.) and sizes are also suitable depending on the application of the nest. FIG. 2 shows one section of the scaffold 30 with two nests 35, 45; however, the scaffold 30 typically has a plurality of nests and preferably, the nests are regularly spaced on the first mesh substrate 32 and the second mesh substrate 41. Also, FIG. 2 shows a three substrate layer scaffold; however, any number of layers of substrate can be used for the scaffold. The nests 35, 45 may be secured to the mesh substrates 32, 41, 51 using various techniques such as adhesive bonding, welding or heat sealing depending on the materials used for the nests and the mesh substrates. The mesh substrates 32, 41, 51 can also be joined by interlocking joints, hinges, or rivets with the three dimensional structure being flexible or locked.

The first mesh substrate 32, the first nest 35, the second mesh substrate 41, the second nest 45, and the third mesh substrate 51 of the scaffold 30 may be formed using the materials and techniques described above with reference to the mesh substrate 12 and the nest 18 of the scaffold 10 of FIG. 1. Also, one or more bioactive agents may be contained within the first nest 35 and/or the second nest 45 of the scaffold 30 as in the nest 18 of the scaffold of FIG. 1. The bioactive agent within each nest may be the same or different depending on the biological activity desired.

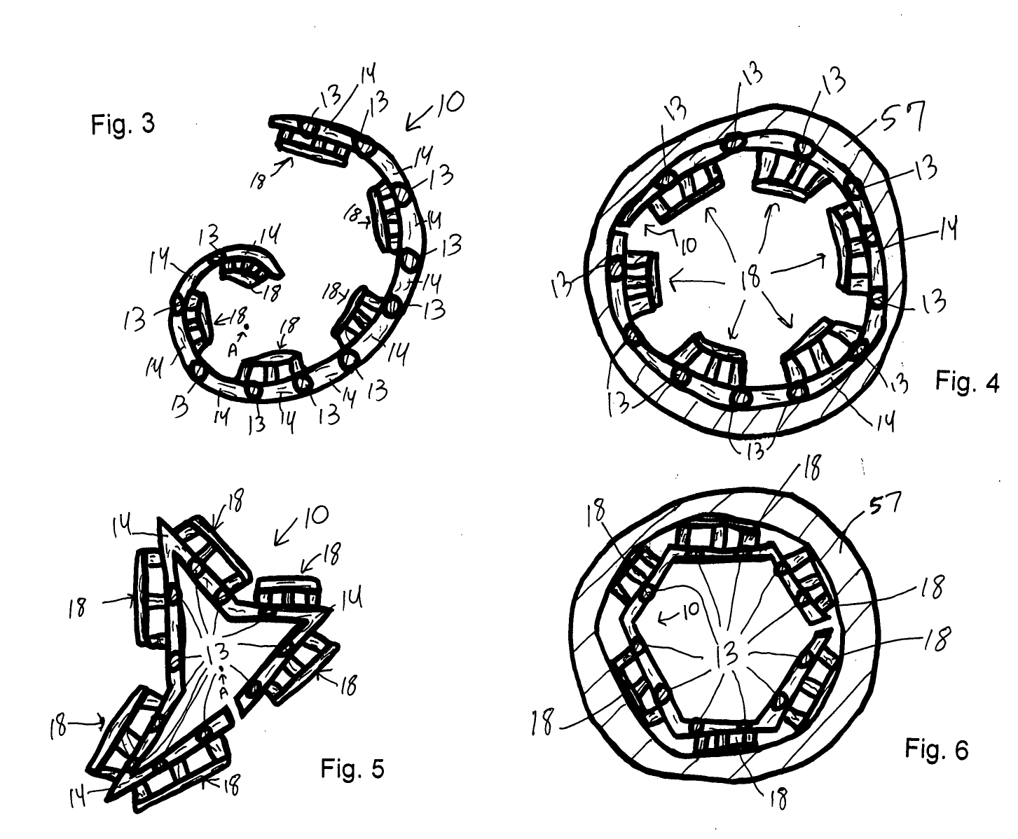

The scaffold 10 of FIG. 1 and the scaffold 30 of FIG. 2 may be formed in a suitable structure (e.g., stent) for location and/or implantation in a body lumen. In order to illustrate this concept, reference is first made to FIG. 3 which shows the example scaffold 10 of FIG. 1 having six nests 18 rolled up around axis A for delivery to a body lumen. The rolled up scaffold 10 can be moved along a guide wire by a catheter in the body lumen (e.g., blood vessel) and expanded by inflation of a balloon within the rolled up scaffold 10. Deflation of the balloon and removal of the guide wire leaves the expanded scaffold 10 in place in the body lumen 57 locked in an expanded state as shown in FIG. 4. The use of a balloon catheter for the location of a stent is known in the art. The scaffold may be rolled up in any configuration that allows the scaffold to be moved in the body lumen for location by expansion.

Turning to FIG. 5, there is also shown the scaffold 10 of FIG. 1 having six nests 18. The example scaffold 10 is inwardly folded at five points toward axis A for delivery to a body lumen. The folded scaffold 10 can be moved along a guide wire by a catheter in the body lumen (e.g., blood vessel) and expanded by inflation of a balloon within the folded up scaffold 10. Deflation of the balloon and removal of the guide wire leaves the expanded scaffold 10 in place in the body lumen 57 locked in an expanded state as shown in FIG. 6. The scaffold may be folded in any configuration that allows the scaffold to be moved in the body lumen for location by expansion.

Other useful structures may be formed from the scaffold 10 or the scaffold 30. For example, the scaffold 10 or the scaffold 30 can be configured as a microscale or nanoscale device for delivery of a bioactive agent to a vessel of a patient such as a body lumen. The microscale or nanoscale devices may be various shapes such as disc, elliptical, spheroid (ball), honeycomb, buckyball-like, or a sphere with regular or irregular (differently sized) depressions. Preferably, the microscale or nanoscale devices are 10-20 microns in size.

The use of magnetic materials in the scaffold 10 or the scaffold 30 can provide advantages when locating or implanting the scaffold 10 or the scaffold 30 in a body lumen. The scaffold 10 or the scaffold 30 can be introduced into a patient's vasculature, and advanced through the vasculature by applying a magnetic field external to the patient in the appropriate direction. If the applied field and the scaffold 10 or the scaffold 30 include a magnetic gradient, the field can be used to advance the scaffold 10 or the scaffold 30 in a desired direction. In one non-limiting example of this technique, a plurality of the scaffolds 10 include a magnetic material and are injected upstream of a vessel closure (e.g. an occlusion). The scaffolds 10 are advanced to the vessel closure by the external magnetic field and the scaffolds 10 pool by the vessel closure and in the capillaries in the area around the closure. The scaffolds 10 have bioactive agents (e.g., cells) seeded therein as described above that would promote angiogenesis in order to bypass the closure. Thus, in one example embodiment, the invention provides a method for treating an occlusion of a blood vessel in a patient.

In another example technique, a plurality of the scaffolds 10 include a magnetic material and the scaffolds are magnetically held against an energized electromagnet at the tip of a catheter. The tip of the catheter is advanced to the vessel closure and the electromagnet is deenergized thereby releasing the scaffolds from the electromagnet of the catheter for pooling by the vessel closure and in the capillaries in the area around the vessel closure. The scaffolds 10 have bioactive agents (e.g., cells) seeded therein as described above that would promote angiogenesis in order to bypass the closure. Thus, this example embodiment of the invention provides another method for treating an occlusion of a blood vessel in a patient. Of course, this technique may be used to treat other conditions.

The use of a targeting moiety, such as a targeting ligand, in the scaffold 10 or the scaffold 30 can also provide advantages when locating or implanting the scaffold 10 or the scaffold 30 in a body location. Targeting ligands can be covalently or non-covalently associated with the scaffold 10 or the scaffold 30. The targeting ligand may be bound, for example, via a covalent or non-covalent bond, to the scaffold 10 or the scaffold 30. The targeting ligands are preferably substances which are capable of targeting receptors and/or tissues in the body. For example, the targeting ligands may be capable of targeting heart tissue and membranous tissues, including endothelial and epithelial cells.

In another example, where a unique cell marker is expressed by a population of cells, such as those making up a tumor, an antibody can be raised against the marker and the antibody can be associated with the scaffold 10 or the scaffold 30. Upon administration of the scaffold 10 or the scaffold 30 to the patient, the binding of the antibody to the marker results in the delivery of the scaffold 10 or the scaffold 30a to the cells (e.g., tumor) whereby the bioactive agent in the scaffold 10 or the scaffold 30 is delivered to the cells (e.g., tumor). Thus, this example method could deliver therapeutic proteins to a tumor. Other non-limiting exemplary targeting agents or moieties include receptors, hormones, adhesion molecules (e.g., lectins, cadherins), or portions or fragments thereof.

The use of electrically charged compounds in the scaffold 10 or the scaffold 30 can also provide advantages when locating or implanting the scaffold 10 or the scaffold 30 in a body location. For example, when cationic compounds are associated with the scaffold 10 or the scaffold 30, ultrasound can be applied to an organ or tissues to deliver the cationic scaffold 10 or the scaffold 30 to the organ or tissues.

The above are non-limiting examples of suitable methods for delivery and targeting of the scaffolds. Other ligands, adhesion molecules, electric charges and magnetic forces can be applied to concentrate and target the scaffolds to a desired site. The same ligands or forces can also be used to anchor these to a delivery device for subsequent timed release.

In one example application of a scaffold according to the invention, the nests include cells such as the smooth muscle progenitor cells described in U.S. Patent Application Publication No. 2004/0247575. In other embodiments, a medical device (e.g., a stent) formed from the scaffold is coated with cells such as the smooth muscle progenitor cells. Smooth muscle progenitor cells can be used to form living vascular grafts, including arterial, venous, and renal grafts or living prosthetic valves for venous and cardiac applications.

For instance, to treat cardiovascular disease, cells can be engineered to produce cell mitogens such as VEGF or FGF-4, ANP, and seeded into the nests of the scaffold, which then is implanted in a patient. In particular, a stent containing cells that secrete VEGF can be used to treat patients with peripheral vascular disease, distal coronary disease, or chronic total occlusions unsuitable for conventional revascularization approaches. Expression of prostacyclin synthase, which produces prostacyclin (PGI₂) from prostaglandin H₂ (PGH₂), in cells can result in delivery of PGI₂ to tissues and can be used for relaxing vascular smooth muscle. Expression of nitric oxide synthase, which catalyzes the production of NO, in cells can result in delivery of NO to tissues and can be used, for example, to inhibit restenosis. Anti-angiogenic polypeptides such as angiostatin and endostatin can be used to aid in the treatment of angiogenic dependent tumors and micrometastases in patients. A similar strategy can be used to aid treatment of biliary duct tumors. Hematopoietic growth factors such as EPO, GM-CSF, and interleukins can be used to increase production of blood cells. For example, EPO can be used to stimulate red cell production and to treat anemia. Thus, a scaffold according to the invention can include cells to implement these example treatment applications.

### INDUSTRIAL APPLICABILITY

The invention provides porous three dimensional structures that may be used to deliver a bioactive agent into a location within the body. In one example form, the porous three dimensional structure may be a stent. In another example form, the porous three dimensional structure may be a microscale or nanoscale device for the delivery of the bioactive agent such as cells.

Although the present invention has been described with reference to certain embodiments, one skilled in the art will appreciate that the present invention can be practiced by other than the described embodiments, which have been presented for purposes of illustration and not of limitation. For instance, while the present invention finds particular utility in coronary applications, there are multiple applications for different organ systems. Therefore, the scope of the appended claims should not be limited to the description of the embodiments contained herein.

What is claimed is:

1. A scaffold for location in a patient, the scaffold comprising:
a substrate;
at least one nest connected to the substrate, the nest extending away from the substrate to define an enclosed volume on the substrate associated with the nest, the nest having openings that extend from an outer surface of the nest to the associated enclosed volume; and
a plurality of cells associated with the scaffold.

2. The scaffold of claim 1 wherein:
the cells are disposed within the enclosed volume on the substrate.

3. The scaffold of claim 1 wherein:
the cells are coated on the scaffold.

4. The scaffold of claim 1 wherein:
the substrate is a flexible mesh.

5. The scaffold of claim 1 wherein:
the scaffold has a plurality of nests.

6. The scaffold of claim 1 wherein:
each nest is rigid.

7. The scaffold of claim 1 wherein:
the substrate has openings.

8. The scaffold of claim 1 wherein:
the substrate is formed from polymeric mesh.

9. The scaffold of claim 1 wherein:
each nest is formed from polymeric mesh.

10. The scaffold of claim 1 wherein:
the substrate is formed from metallic wire cloth.

11. The scaffold of claim 1 wherein:
each nest is formed from metallic wire cloth.

12. The scaffold of claim 1 wherein:
the openings of the nest are least 60 microns.

13. A medical device selected from the group consisting of grafts, pledges, artificial ureters, shunts, cartilage, dura, tympanic membrane, biliary duct, skin, biological pacemaker wires, leads, heart valves, nerve fibers, and aneurysm coils or stents, wherein the medical device comprises the scaffold of claim 1.

14. A scaffold for location in a patient, the scaffold comprising:
a non-woven substrate;
at least one non-woven nest connected to the substrate, the nest extending away from the substrate to define an enclosed volume on the substrate associated with the nest, the nest having openings that extend from an outer surface of the nest to the associated enclosed volume; and
a bioactive agent disposed within the enclosed volume on the substrate.

15. The scaffold of claim 14 wherein:
the substrate is a flexible mesh.

16. The scaffold of claim 14 wherein:
the nest includes a side wall and a top wall, and
the top wall of the nest is part of a second substrate, and
the side wall of the nest has openings extending through the side wall.

17. The scaffold of claim 16 wherein:
at least one nest is connected to the second substrate, the nest connected to the second substrate extending away from the second substrate to define an enclosed volume on the second substrate associated with the nest connected to the second substrate, the nest connected to the second substrate having openings that extend from an outer surface of the nest to the associated enclosed volume; and
a bioactive agent disposed within the enclosed volume on the second substrate.

18. The scaffold of claim 17 wherein:
the nest connected to the second substrate includes a side wall and a top wall, and
the top wall of the nest connected to the second substrate is part of a third substrate, and
the side wall of the nest connected to the second substrate has openings extending through the side wall.

19. The scaffold of claim 14 wherein:
the scaffold has a plurality of nests.

20. The scaffold of claim 14 wherein:
each nest is rigid.

21. The scaffold of claim 14 wherein:
the substrate has openings.

22. The scaffold of claim 14 wherein:
the substrate comprises a material selected from polymeric materials and metallic materials.

23. The scaffold of claim 14 wherein:
the openings of the nest are least 60 microns.

24. The scaffold of claim 14 wherein:
the scaffold has a plurality of nests and the nests are regularly spaced on the substrate.

25. The scaffold of claim 14 wherein:
the bioactive agent is selected from cells, precursors, drugs, enzymes, organic catalysts, ribozymes, organometallics, proteins, glycoproteins, peptides, polyamino acids, antibodies, nucleic acids, steroidal molecules, antibiotics, antimycotics, cytokines, growth factors, carbohydrates, oleophobics, lipids, extracellular matrix and/or its individual components, pharmaceuticals, therapeutics, and mixtures thereof.

26. The scaffold of claim 14 wherein:
the bioactive agent is selected from cells, precursors, and mixtures thereof.

27. A medical device selected from the group consisting of grafts, pledges, artificial ureters, shunts, cartilage, dura, tympanic membrane, biliary duct, skin, biological pacemaker wires, leads, heart valves, nerve fibers, and aneurysm coils or stents, wherein the medical device comprises the scaffold of claim 14.

28. A device for delivery of a bioactive agent to a vessel of a patient, the device comprising:
a substrate;
at least one nest connected to the substrate, the nest extending away from the substrate to define an enclosed volume on the substrate associated with the nest, the nest having openings that extend from an outer surface of the nest to the associated enclosed volume; and
a bioactive agent disposed within the enclosed volume on the substrate,
wherein the device is a nanoscale device.

29. The device of claim 28 wherein:
the substrate is a flexible mesh.

30. The device of claim 28 wherein:
the bioactive agent is selected from cells, precursors, and mixtures thereof.

31. The device of claim 28 wherein:
at least one nest or the substrate comprises a magnetic material.

32. A method for treating an occlusion of a blood vessel in a patient, the method comprising:
injecting a plurality of the device of claim 28 upstream of the occlusion such that the plurality of the devices locate near the occlusion.

33. A method for treating an occlusion of a blood vessel in a patient, the method comprising:
injecting a plurality of the device of claim 31 in the blood vessel; and
moving a magnetic field external to the patient such that the plurality of the devices are advanced to and locate near the occlusion.

34. A method for treating an occlusion of a blood vessel in a patient, the method comprising:
magnetically adhering a plurality of the device of claim 31 to a catheter;
moving the catheter in the blood vessel such that the plurality of the devices are located near the occlusion; and
releasing the plurality of the devices from the catheter.

35. A method for delivering a bioactive agent to tissue in a patient, the method comprising:
administering to a site in the patient a plurality of the device of claim 28 such that the plurality of the devices locate near the tissue.

36. The method of claim 35 wherein the device includes a targeting moiety that binds with a moiety of the tissue.

37. The method of claim 36 wherein the targeting moiety is selected from the group consisting of ligands, antibodies, receptors, hormones, adhesion molecules, or portions or fragments thereof.

38. The method of claim 35 further comprising:
applying a magnetic field near the tissue such that the plurality of the devices locate near the tissue.

39. The method of claim 35 further comprising:
applying an electrical field near the tissue such that the plurality of the devices locate near the tissue.

40. A radially expandable lumenal scaffold comprising:
a deformable substrate;
at least one nest connected to the substrate, the nest extending away from the substrate to define an enclosed volume on the substrate associated with the nest, the nest having openings that extend from an outer surface of the nest to the associated enclosed volume; and
a bioactive agent disposed within the enclosed volume on the substrate,
wherein the substrate is wrapped about an axis to form the scaffold.

41. The scaffold of claim 40 wherein:
the nest extends away from the substrate toward the axis.

42. The scaffold of claim 40 wherein:
the nest extends away from the substrate away from the axis.

43. The scaffold of claim 40 wherein:
the scaffold has a plurality of nests.

44. A radially expandable lumenal scaffold comprising:
a deformable substrate;
at least one nest connected to the substrate, the nest extending away from the substrate to define an enclosed volume on the substrate associated with the nest, the nest having openings that extend from an outer surface of the nest to the associated enclosed volume; and
a bioactive agent disposed within the enclosed volume on the substrate,
wherein the substrate is folded to form the scaffold.

45. The scaffold of claim 44 wherein:
the nest extends away from the substrate toward the axis.

46. The scaffold of claim 44 wherein:
the nest extends away from the substrate away from the axis.

47. The scaffold of claim 44 wherein:
the scaffold has a plurality of nests.
